(19) United States
(12) Patent Application Publication
(10) Pub. No.: US 2003/0134345 A1
(43) Pub. Date: Jul. 17, 2003

(54) Serological assay for detection of antigens sequested within immune complexes

(76) Inventor: Michael Brunner, Columbus, NJ (US)

Correspondence Address: PERKINS COIE LLP, POST OFFICE BOX 1208, SEATTLE, WA, 98111-1208, US

(21) Appl. No.: 10/197,114

(22) Filed: Jul. 17, 2002

Related U.S. Application Data
(60) Provisional application No. 60/305,933, filed on Jul. 17, 2001.

(51) Int. Cl. G01N 33/554; G01N 33/569

(52) U.S. Cl. 435/7.32

(57) ABSTRACT

A method for detecting an antigen present in an immune complex comprises isolating an immune complex, which contains at least one antigen and at least one antibody specific for that antigen, from a sample, then incubating the immune complex under conditions effective to dissociate the immune complex and separate the antigen from the antibody. The dissociated antigen and antibody are then reassociated, and the reassociated antigen and antibody are separated from the solution, using a binding agent on a solid phase. The presence of the antigen is then detected and optionally quantified using an antigen-specific binding reaction. A second method is provided, in which the undissociated immune complexes are bound to a solid surface through an anti-IgM antibody, then are incubated with a biotinylated extract containing an antigen specific for the immune complex antibody. The presence of the biotin molecule is detected and optional quantified using a biotin-specific binding reaction.

This application claims the priority of U.S. Provisional Patent Application Serial No. 60/305,933, filed Jul. 17, 2001, the entirety of which is hereby incorporated by reference.

### FIELD OF THE INVENTION

The present application is directed to serological assays for the diagnosis of infectious diseases in patients by detecting infectious disease-specific antigens, or the antibodies against such antigens, sequestered within immune complexes (ICs). The present application is also directed to serological assays for the diagnosis of autoimnmune diseases in patients by detecting autoimmune disease-specific antigens sequestered within ICs.

### BACKGROUND OF THE INVENTION

Lyme disease is a potentially multi-system inflammatory disease in humans caused by the spirochete, Borrelia burgdorferi, which infects through the bite of spirochete-infected ticks. Lyme disease is often diagnosed clinically by the presence of bullseye-shaped erythema migrans (EM) rash. However, this “bullseye rash” occurs in only 60% to 80% of patients, and can be atypical in appearance. At about the same time that the rash develops, flu-like symptoms, such as headache, sore throat, stiff neck, fever, muscle aches, fatigue and general malaise, may appear. In some cases, the flu-like illness may appear without any rash.

If ignored, the early symptoms may disappear, but more serious problems can develop months or years later. The later symptoms of Lyme disease can be quite severe and chronic. Neurological symptoms (i.e., meningitis, numbness, tingling, and burning sensations in the extremities), Bell's palsy, severe pain and fatigue, and depression may also occur. Heart, eye, respiratory and gastrointestinal problems can develop. Muscle pain and arthritis, usually of the large joints, is common. Symptoms are often intermittent, lasting from a few days to several months and sometimes years. Because of its diverse symptoms, chronic Lyme disease mimics many other diseases and therefore can be difficult to diagnose in the absence of the bullseye rash.

A diagnostic gold standard for Lyme disease is a positive culture from a biopsy (usually skin) of the affected area, in which Borrelia burgdorferi is detected. However, this test is difficult to perform, expensive, and requires specialized testing facilities. Direct detection of B. burgdorferi infection from biopsies by other means, such as detecting B. burgdorferi antigens by immunohistochemistry or identifying B. burgdorferi DNA by polymerase chain reaction (PCR), are also technically demanding and problematic. As a result, biopsy culture is not a practical approach for broad scale testing.

Although the diagnosis of Lyme disease is most accurate when based on objective historical and clinical findings, serological evidence may be useful or necessary in some circumstances. In the presence of EM, serologic confirmation is not necessary. However, lacking an EM lesion, an isolated “viral syndrome” following a tick bite cannot be attributed to early B. burgdorferi infection without laboratory confirmation. The early features of Lyme disease may occur too soon after tick bite for specific humoral immune responses to be detectable by current immunoassays.

Indirect measurements are often used to confirm a diagnosis of B. burgdorferi infection. Such indirect measurement techniques include detection of anti-B. burgdorferi antibodies in serum or other bodily fluids (e.g., synovial or cerebrospinal fluid) by an enzyme-linked immunosorbent assay (ELISA) and/or an immunoblot (Western blot). Serum IgM anti-B. burgdorferi antibodies are often detectable in the early stages of Lyme disease; by 6 to 8 weeks IgG is detectable in the majority of untreated patients. However, early serologic confirmation of Borrelia burgdorferi infection can be problematic, because specific antibodies to B. burgdorferi antigens are usually bound up in circulating immune complexes, rendering the antibodies unavailable or undetectable by standard techniques. Immune complexes (ICs) are large lattice networks of interlocking antigens and antibodies. Usually, the body is able to clear ICs from the circulation, but under some conditions ICs continue to circulate in the blood. In fact, elevated levels of circulating ICs was one of the earliest described immunologic phenomena in Lyme disease. Consequently, antibodies are not present in the circulation initially. Since routine indirect serodiagnostic tests rely on detecting antibody free in the serum or other bodily fluid, those antibodies tied up in complexes are unavailable and therefore missed, producing false negative results. The clinical features of Lyme disease may develop before anti-B. burgdorferi antibodies become detectable in bodily fluids.

On the other hand, even after successful antibiotic treatment, anti-B. burgdorferi antibody titers can remain elevated for years post-infection. Persistent seropositivity may be incorrectly interpreted as indicating ongoing infection. The frequency of false positive ELISA results thus dictates a two-tier strategy, with an ELISA or an immunofluorescent assay (IFA) of a serum sample as a screen, followed by immunoblot confirmation of a positive or equivocal ELISA. Known consensus bands for IgM (early cases) or IgG that are specific for B. burgdorferi antigens are ascertained before diagnosis is confirmed. Criteria for immunoblot interpretation are widely accepted, but testing is not standardized and may still generate a high number of false positive results due to the presence of cross-reactive antigens or antibodies, such as rheumatoid factor, the 23 kDa antigen from Helicobacter pylori, or the flagellin 41 kDa antigen of Treponema pallidum. Hence, Lyme disease can still be easily over-diagnosed, even when the two-tier diagnostic strategy is used.

In addition to B. burgdorferi infection that leads to Lyme disease, other organisms which can generate immune complexes upon infection include Treponema pallidum, the causative agent of syphilis, and Streptococcus pyogenes, which can cause glomerulonephritis. Immune complexes are also a feature of a variety of autoimmune diseases. In these diseases, immune complexes may form as complexes of autoantibodies and intracellular antigens, and accumulate in kidneys, lung, skin, joints, and/or blood vessels. There the complexes set off reactions that lead to inflammation and tissue damage. While the classical example of an immune complex-mediated autoimmune condition is systemic lupus erythematosus (SLE), other autoimmune conditions, such as multiple sclerosis, glomerulonephritis, gout, rheumatoid arthritis and polyarteritis nodosa, also produce immune complexes. As with B. burgdorferi infection, sequestration of antigens and autoantibodies in immune complexes leads to difficulties in early diagnosis of the autoimmune condition.

### SUMMARY OF THE INVENTION

In one embodiment, a method for detecting an antigen present in an immune complex comprises isolating an immune complex from a bodily sample derived from a patient, the immune complex including an antigen and an antibody directed against the antigen; incubating the immune complex under conditions effective to dissociate the immune complex and separate the antigen from the antibody; incubating the dissociated immune complexes under conditions effective to reassociate the antigen and the antibody; incubating the reassociated antigen and antibody with at least one member selected from the group consisting of protein A-agarose beads, protein G-agarose beads, protein L-agarose beads, mannan-binding protein-agarose beads, and agarose beads containing at least two of protein A, protein G, protein L and mannan-binding protein, under conditions effective to permit binding of the antibody by the protein on the agarose beads; separating the agarose beads from the solution; and detecting the presence of the antigen bound to the agarose beads through a binding reaction specific for the antigen.

In another embodiment, a method for detecting an antibody present in an immune complex comprises isolating an immune complex from a bodily sample derived from a patient, the immune complex including an antigen and an antibody directed against the antigen; incubating the immune complex under conditions effective to dissociate the immune complex and separate the antigen from the antibody; incubating the dissociated immune complexes with a solid phase bearing an anti-IgM antibody under conditions effective to permit binding of the antibody of the immune complex by the anti-IgM antibody; incubating the solid phase with a biotinylated sonicate of Borrelia burgdorferi cells; and detecting the presence of the antibody bound to the solid phase through a binding reaction specific for the biotinylated sonicate.

The above and other objects, features and advantages of the present invention will become apparent from the following description read in conjunction with the accompanying drawings.

### BRIEF DESCRIPTION OF THE DRAWINGS

FIG. 1 shows Western blots using anti-OspA antibody H5332 to detect Osp A protein in immune complexes derived from various Lyme disease patients BEADS METHOD (row I, lanes 1-4; row II, lanes 2, 3, 5-8; row III, lanes 1-8) and Lyme disease-negative controls (row 1, lanes 5-8; row II, lane 4). Rows I-III, lane 9; row II, lane 1—B. bergdorferi positive controls. Row I, lane 1—serum sample from patient with biopsy-culture positive EM lesion, 7 days after biopsy. Lane 2—same as lane 1, 9 days after biopsy. Lane 3—same as lane 1, 3 days after biopsy. Lane 4-7 days after biopsy. Row II, lane 2—EM(+) patient with tertiary neuroborreliosis. Lane 3—patient with Lyme arthritis. Lane 5—patient with tertiary neuroborreliosis. Lane 6—serum sample from patient with biopsy-culture positive EM lesion, 9 days after biopsy. Lane 7—EM(+) patient. Lane 8—patient with tertiary neuroborreliosis. Row III, lane 1—EM(+) patient. Lane 2—EM(+) patient with seventh nerve palsy. Lane 3—EM(+) patient with tertiary neuroborreliosis. Lane 4—patient with lymphocytic meningitis. Lane 5—patient with tertiary neuroborreliosis. Lane 6—patient with Bannworth syndrome. Lane 7—patient with Lyme arthritis. Lane 8—serum sample from patient with biopsy-culture positive EM lesion, 9 days after biopsy.

FIG. 2 shows OspA detection in immune complexes of a seronegative, culture positive, EM positive Lyme disease patient. A Western blot was performed using the OspA-specific monoclonal antibody, H5332. Lane 1—Borrelia burgdorferi sonicate. Lane 2—agarose immunobead eluate. Lane 3—dissociated immune complex.

FIG. 3 is a comparison of serum with immune complex derived IgM, in the enzyme-linked IgM capture immune complex biotinylated antigen assay (“EMIBA”). Samples A-E—different patients with active Borrelia burgdorferi infection. Patients A and C had tertiary neuroborreliosis. Patient B had Lyme arthritis. Patients D and E were EM(+). Patient F was a Lyme disease(−) control patient. Patient G was a patient with past Lyme disease who had been treated and cured with antibiotic therapy. Patient G was asymptomatic and without clinical evidence of active infection at time sample was obtained.

### DETAILED DESCRIPTION OF THE PREFERRED EMBODIMENTS

In one embodiment, the present application discloses an assay for the detection of one or more antigens complexed to antibodies within an immune complex (IC). This assay unambiguously isolates and confirms the presence of an infecting organism, using a sample derived from a bodily fluid. More specifically, the assay isolates and confirms the presence of antigens derived from the infecting organism that are found in immune complexes. The present method is able to distinguish between seropositivity due to active infection and seropositivity due to prior, treated disease. Furthermore, the method is able to detect antigens of infectious organisms in otherwise seronegative patients, thereby enabling earlier detection and treatment of such conditions.

In a second embodiment, the present application discloses an assay, designated the EMIBA (Enzyme-linked, IgM-capture, Immune complex, Biotinylated antigen Assay) for the detection and quantification of immune complexes (ICs). The EMIBA reduces or eliminates interference from extraneous serum components that may interfere with the assay or produce a false positive result. Because it binds to antibodies derived only from immune complexes, the assay is sufficiently sensitive to confirm early infection; sufficiently specific to obviate the requirement for immunoblot confirmation; and capable of differentiating active infection from persisting seropositivity in patients with successfully treated disease.

In both cases, a sample is first treated to precipitate immune complexes. The sample may be obtained from a human patient, and may be blood, serum, cerebrospinal fluid, pericardial fluid, or tissue, or samples derived from one or more of the above. Precipitation of the immune complexes is accomplished by incubation of the sample with an amount of polyethylene glycol (PEG) that is effective to render the immune complexes insoluble in the sample solution, for a period of time between 2 hr and overnight. The precipitated complexes are collected by centrifugation, and then dissociated by incubation in a high pH buffer. As an example of such a buffer, 0.1 M sodium borate buffer, pH 10.2 may be used. However, any buffer with a significant buffering capacity in the range between 8.0 and 14.0 may be used. Thereafter, the solution containing dissociated immune complexes is neutralized by addition of a neutral or acid buffer, such as potassium or sodium acetate, pH 5.3, to a final pH of between 6.5 and 8.0. Alternatively, in lieu of dissociating immune complexes in a high pH buffer followed by neutralization, a neutral or nearly neutral buffer, such as PBS, may be used to dissociate the immune complexes. However, such buffers are not as effective at dissociating the immune complexes as are the higher pH buffers.

In the first embodiment, the dissociated immune complexes are treated to isolate the antigens present in the ICs by separation of antibody/antigen complexes present in the ICs from the rest of the IC components. After dissociation, the immune complex components are incubated with agarose beads bearing one or more proteins that bind to immunoglobulins. As examples, there can be mentioned protein A, protein G, protein L and mannan-binding protein. Protein A binds to IgG from a number of species, as well as human IgM, IgA and IgE. Protein G binds to IgG molecules from a wide variety of species. Protein L specifically binds most forms of immunoglobulin molecules that contain K light chains, including IgG, IgM, IgA, IgD, IgE, and IgY, from human, mouse, rat, rabbit, and chicken. Mannan-binding protein beads specifically bind mouse and human IgM. These proteins bind to the immunoglobulins without interfering with the antigen-binding domain, and therefore can be used to bind antibody/antigen complexes as well. Incubation of the immune complex components with beads bearing these proteins will capture both free antibodies and antibody/antigen complexes derived from the immune complexes. In one embodiment, the immune complex components are incubated with beads bearing one of protein A, protein G, protein L and mannan-binding protein. In another embodiment, the beads may bear more than one of protein A, protein G, protein L and mannan-binding protein. In another embodiment, the immune complex components are incubated sequentially, in any combination, with beads bearing one of protein A, protein G, protein L or mannan-binding protein. In yet another embodiment, the immune complex components may be incubated sequentially, in any combination, with beads bearing at least one of protein A, protein G, protein L or mannan-binding protein.

After incubation, the beads are separated from the immune complex solution by centrifugation, filtration, or by merely allowing them to settle at normal gravity. The beads then are washed and analyzed by SDS-PAGE and Western blot, as is known in the art. The Western blot may probe using an antibody specific for an antigen component of the immune complex. In another embodiment, the Western blot may be probed with an antigen or antibody specific for an antibody component of the immune complex. In another embodiment, the beads may be incubated under conditions that release the bound antigen or antibody, then the eluate exposed to a solid phase bearing an antibody to an antigen or antibody component of the immune complex. Such a solid phase can take the form of, for example, a dipstick or slide.

In a second embodiment, designated the EMIBA, a solid-phase reaction surface is coated with anti-human IgM antibody. The reaction surface may be any appropriate surface useful in the chosen assay format, i.e., a plate or slide made of plastic, paper, glass, or some combination of these. After the solid-phase reaction surface has been coated with the anti-human IgM antibody, the surface is washed and blocked. Then, the surface is contacted with a biotinylated serum sample or a sample containing wholly or partially dissociated immune complexes. After incubation and washing, the solid-phase reaction surface is contacted with a reporter molecule linked to an anti-biotin antibody or antibody fragment, or to an avidin molecule or avidin fragment. The solid-phase reaction surface is then developed as appropriate for the reporter molecule.

In a third embodiment, instead of using a solid-phase reaction surface coated with anti-human IgM antibody, the dissociated ICs are first treated with agarose beads bearing one or more proteins that bind to immunoglobulins. The beads are incubated with the dissociated ICs in a solution, and preferably are mixed during the incubation step to ensure that the sample is completely exposed to the beads. After incubation, the beads are separated from the solution by centrifugation, filtration, or by merely allowing them to settle at normal gravity. After incubation and washing, the solid-phase reaction surface is contacted with a reporter molecule linked to an anti-biotin antibody or antibody fragment, or to an avidin molecule or avidin fragment. The solid-phase reaction surface is then developed as appropriate for the reporter molecule.

The anti-human IgM antibody may be polyclonal or monoclonal, and may be obtained from a variety of commercial sources. The anti-human IgM antibody may be derived from any appropriate organism, such as mouse, rat, rabbit, monkey, hamster, chicken, or even human. The anti-human IgM antibody may be any form of antibody known to those of skill in the art, including IgG, IgM, IgA, IgE, F(ab′)₂, Fab′, Fab, or Dab. The antibodies further may be humanized or bispecific.

The reporter molecule likewise may be any appropriate molecule, including but not limited to an enzyme, a chromophore, a fluorophore, or a radioactive molecule. Examples of reporter molecules that are enzymes include β-galactosidase, alkaline phosphatase, peroxidase, urease, β-glucuronidase, β-D-glucosidase, β-D-galactosidase, luciferase, and horseradish peroxidase. Examples of reporter molecules that are fluorescent dyes include fluorescein, rhodamine, auramine, or Texas Red. Examples of radioactive molecules include ¹⁴C, ¹²⁵I, ¹³¹I, ³²P, ³³P, ³⁵S, and ³H.

Materials and Methods

Serum and Plasma collection: For serum samples, blood was drawn and allowed to clot at room temperature for 1 hour; for plasma, heparinized blood was left at room temperature for 1 hour. Both types of specimens were then centrifuged in a centrifuge (Sorvall Model RC5C, Sorvall-DuPont, Wilmington, Del., HS-4 rotor) at 769×g for 10 minutes. Clear serum or plasma were drawn off by pipette. After analysis, serum and plasma were stored at −70° C. until needed for further testing. Freeze/thaw only slightly decreased reactivity of some samples.

Immune complex precipitation: The polyethylene glycol (PEG) method was used to isolate immune complexes (ICs). Samples were precipitated with an equal volume of 0.1 M sodium borate buffer, pH 8.4, containing 7% PEG (average molecular weight 8,000, Sigma Chemical Co., St. Louis, Mo. (hereinafter “Sigma”)) and 0.44% NaCl. The tubes were vortexed, left at 4° C. for at least 4 hours, and centrifuged at 10,000 rpm (8,320×g) for 15 minutes 4° C. Supernatants were carefully removed with a pipette. The pellet was resuspended and washed twice with 200 μl of a 3.5% PEG solution in the same buffer. After the second spin, samples were resuspended in 100 μl 0.1 M sodium borate buffer, pH 10.2; high pH buffer is better than PBS for dissociating immune complexes and does not affect antibody stability. Dissociated immune complexes were kept in buffer at 4° C. until use. There was no discernible loss of reactivity after storage for 2 weeks. For the precipitation of B. burgdorferi antigens, the dissociated ICs were neutralized with 3M sodium acetate buffer, pH 5.3, to reassociate the antigen/antibody complexes, and stored as above.

Immunoprecipitation Using Agarose Beads Coated with Antibody-Binding Proteins: Gammabind® G Sepharose (Pharmacia Corp., Piscataway, N.J.) was added to a sample of reassociated ICs and placed on a reciprocal shaker at 4° C. for 1 hr. Thereafter, the process was repeated by addition of Ultralink® agarose beads containing mannan binding protein (Pierce Chemical Co., Rockford, Ill. (hereinafter “Pierce”)) to remove all complexes containing IgM, followed by addition of protein L-agarose (Santa Cruz Biotechnology, Santa Cruz, Calif.) to bind all the remaining IgM, IgG, and IgA antibodies. The beads were shaken overnight at 4° C., followed by centrifugation at 10,000 RPM (8,600×g) for 15 minutes. The supernatant was aspirated, and thereafter the bead pellet was resuspended in a reducing buffer, and boiled to release antigen. A portion of the supernatant then was electrophoresed through 12% acrylamide gels and transferred to a Polyscreen® PVDF membrane (NEN, Boston, Mass.). The membrane was then probed with anti-OspA monoclonal antibody H5332 to detect the 31 KDa OspA protein. The secondary antibody was peroxidase-labeled goat anti-mouse IgG (Fab specific) (Sigma), and visualization was accomplished using Renaissance Plus™ enhanced luminol chemiluminescent substrate (NEN, Boston, Mass.).

Preparation of B. burgdorferi sonicate: High passage B. burgdorferi strain B-31 was grown in BSK medium (Sigma) supplemented with 6% normal rabbit serum (Gemini Bio-Products, Calabasas, Calif.), in T-flasks (Corning Glass, Corning, N.Y.) at 32° C. 400 ml of late log phase culture was harvested by centrifugation at 9,000×g for 15 minutes, and washed 3 times with cold phosphate buffered saline (PBS), pH 7.2. The final pellet was resuspended in 2 ml of PBS and sonicated (Braun-Sonic 2000, Kronberg, Germany) on a medium setting for 4 thirty-second pulses, with one minute rests between pulses. Approximately 9 mg/ml of protein was obtained, assayed by BCA protein assay (Pierce) and stored at −70° C. until use. In certain studies a low-passage B. burgdorferi N40 strain was propagated and processed as above. The results shown herein were obtained using the B. burgdorferi B31 strain, since this is the organism used in the commercial ELISA and immunoblot kits used for comparison. In side-by-side studies, results obtained using N40 and B31 strains were identical.

Biotinylation of B. burgdorferi sonicate: Different long-arm biotin hydroxysuccinamide esters, including biotinamidocaproate N-hydroxysuccinamide (NHS) ester (Sigma) and NHS-LC-biotin II (Pierce) were equally effective; studies described in this paper used biotinamidocaproate N-hydroxysuccinamide. One ml of a 9 mg/ml protein sonicate solution was adjusted to pH 9 by adding 0.1 volume 0.5M carbonate/bicarbonate buffer pH 9 immediately before biotinylation. Optimal biotinylation was achieved using 50 mg/ml biotin ester in dimethylformamide (DMF). To the pH-adjusted protein sonicate, 20 μl of biotin solution was added in a 16×100 mm glass tube, covered, and slowly rotated for 1-2 hours at room temperature, pipetting up and down every 15 min. The reaction was stopped by adding 0.1 vol 1M Tris-HCl pH 7.5. Biotinylated B. burgdorferi sonicate was dialyzed against 3 changes (2 liter each) of 20 mM PBS, pH 7.2, containing 0.05% sodium azide in the cold using a Slide-A-Lyzer (Pierce) with a 10 kDa cut-off. The resulting suspension was assayed for protein, using the BCA protein assay reagent (Pierce), aliquoted and frozen at −70° C. Typical yields were at least 75%.

EMIBA and Free Antibody Tests: Affinity purified goat anti-human IgM (μ chain specific) (100 μl/well, KPL, Gaithersburg, Md.), at a concentration of 10 μg/ml in 40 mM/35.7 mM carbonate/bicarbonate coating buffer, pH 9.6, was added to Immulon 4 microtiter plates (Dynatech Laboratories, Chantilly, Va.). Plates were rotated slowly at room temperature for 2 hours and stored covered at 4° C. overnight. The plates were then warmed to room temperature and washed 3 times with 10 mM PBS, pH 7.5, containing 0.1% BSA (Sigma) and 0.05% Tween-20 (PBS-BT) using a Bio-Tek ELP 35 automated plate washer (Bio-Tek Instruments, Inc., Winooski, Vt.). After the final wash, 0.35 ml per well of a blocking buffer (PBS-BT containing 5% non-fat dry milk) was added, the plates covered with Mylar, incubated for 1 hour at 37° C. and washed twice with PBS-BT. 100 μl/well of dissociated immune complexes (EMIBA) at 1:10 dilution or serum at 1:100 dilution in PBS-BT with 3% fish skin gelatin (Sigma) and 1% heat-inactivated normal goat serum (Vector Labs, Burlingame, Calif.) (free antibody assay), was added in duplicate. Plates were incubated for 2 hours, washed three times with PBS-BT and biotinylated B. burgdorferi (prepared as described above) was added. The plate was covered and rotated slowly for ½ hour at room temperature.

After three washes with PBS-BT, a 1/8,000 dilution of peroxidase-labeled goat anti-biotin (Vector Labs, Burlingame, Calif.) in incubation buffer was added, covered, slowly rotated for hour and washed on a plate washer for 3 cycles with PBS-BT, followed by two manual PBS washes. 100 μl/well of a two component 3,3′5,5′ tetramethylbenzadine (TMB) substrate solution (KPL, Gaithersburg, Md.) was added. The plates were tapped lightly several times, observed for color development and the reaction stopped after 10 minutes with 100 μl 1M H₃PO₄. The optical density values were calculated as the average OD of duplicate samples. The assays were developed so that the same positive controls in each run gave an OD of approximately 1.0, negative controls an OD of less than 0.1, and wells coated with the anti-IgM with no serum added gave an OD of 0.05 or less when read at dual wavelengths on a Bio-Tek EL312E ELISA plate reader (signal at 450 nm, background at 630 nm). The optimal amount of biotinylated B. burgdorferi was determined for each batch (4 to 12 μg/ml), each batch was standardized with the preceding preparation using the same positive and negative panels. Optimal dilution for dissociated immune complex was 1/10, and for serum was 1/100.

The positive cut-off for each plate was the mean of 10 negative control samples run in duplicate plus 3 standard deviations. Normal control sera were obtained from the University Diagnostic Laboratory of Robert Wood Johnson Medical School (RWJMS); all subjects resided in New Jersey or the surrounding area and were without known history of Lyme disease. Dividing the average OD of the patient sample by the cut-off gave an index value. Index values equal to or greater than 1.0 were considered positive; less than 1.0 was negative.

Statistical Analysis: Based on “active” or “prior” status of Lyme disease at the time of phlebotomy, test results were designated as True Positive (TP), True Negative (TN), False Positive (FP) or False Negative (FN). Sensitivity and specificity were calculated as follows: Sensitivity=[TP/(TP+FN)]×100; Specificity=[TN/(TN+FP)]×100. The 95% confidence intervals for sensitivity and specificity were calculated using the Fleiss correction (Fleiss, J., Statistical Methods for Rates and Proportions, 2^{nd} Ed., p. 328, John Wiley & Sons, Hoboken, N.J. (1981)). Significance of differences in the sensitivity and specificity of the different assays tested were assessed by McNemar's Chi square test (Siegel, S. and N. Castellan, Nonparametric Statistics for the Behavioral Sciences, 2^{nd} d Ed., p. 75-80, McGraw-Hill, New York, N.Y. (1988)). Also provided are 95% confidence intervals of differences between the sensitivities and specificities of the different assays. As collections were stratified into “active” and “prior” groups, the number of samples within some cells was too small to calculate confidence intervals.

### EXAMPLE 1

Detection of Lyme Disease Proteins Using Agarose Beads Coated with Immunoglobulin-Binding Proteins

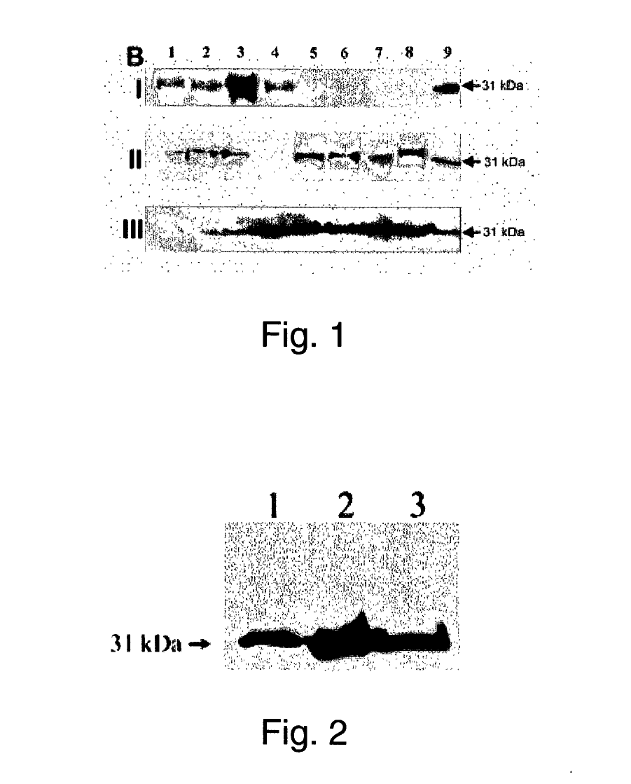

Immunoprecipitation of partially concentrated PEG precipitates with human Fc-binding protein bead conjugates yielded a preparation that contained a 31 kDa protein bound by H5332, a monoclonal antibody against OspA, in the immunoblot (FIG. 1). OspA was found in immune complexes from seropositive and culture positive patients (row I, lanes 1 to 4), but not from negative (seronegative) controls (row I, lanes 5 to 8). No reactivity was detected by monoclonal antibody H9724 (anti-flagellin 41 kDa antigen) or with L22 1F8 (anti-OspC) (not shown). H5332 was reactive with the OspA of sonicated Borrelia burgdorferi (row I, lane 9). OspA was detected in patients with central nervous system Lyme disease (row II, lanes 2, 5 and 8). Lyme arthritis (row II, lane 3) and culture positive erythema migrans (row II, lane 6). OspA was found in patients with Lyme disease and multiple erythema migrans (row III, lanes 1 and 3), culture positive erythema migrans (row III, lane 8), seventh nerve palsy (row III, lane 2), lymphocytic meningitis (row III, lane 4), encephalopathy (anti-Borrelia burgdorferi antibodies present in CSF, row III, lane 5), Bannwarth syndrome (lymphocytic meningitis, seventh nerve palsy and radiculoneuritis) (row III, lane 6), and Lyme arthritis (row III, lane 7).

These results are representative of multiple analyses that used different serum samples from patients with different features of Lyme disease—for example, tertiary neuroborreliosis and Lyme arthritis—as well as from relevant controls. Most patients with early Lyme disease had immune complexes containing detectable OspA. Half the patients with later features of Borrelia burgdorferi infection had OspA within their immune complexes, including 3 of the 4 patients with tertiary neuroboreliosis, and 1 of the 4 patients with arthritis. None of the 37 serum samples from controls contained material released from their immune complexes that was detected by H5332, the anti-OspA monoclonal antibody used in these experiments.

The serum from a patient who was seronegative on standard Lyme assays, including ELISA, IFA, and immunoblot, but who presented with EM and was culture positive from biopsy taken at the time of the serum sample, was tested for Lyme antigen as part of IC. The patient's serum was first PEG precipitated at a concentration known to bring down high molecular weight circulating immune complexes (if present) but not free antibodies. When the dissociated PEG precipitate of the patient's serum was first treated with antibody-binding beads, boiled to release antigen which was in a complex with the antibody, and probed with monoclonal antibody H5332, a band with the same electrophoretic mobility as the 31 kD OspA antigen was observed (FIG. 2, lane 2). A single band was obtained when a B. burgdorferi sonicate was used as the starting material (lane 1). When dissociated immune complex was electrophoresed, blotted and probed with monoclonal antibody against OspA (H5332), a single 31 kD band was obtained (lane 3). Therefore, Lyme-specific antigen (OspA) complexed to antibody was detectable in the serum of a patient with Lyme disease at a time when routine Lyme testing (by ELISA, IFA, and immunoblot) was negative. This example provides further proof that the 31 kDa band detected by the H5332 antibody is present in an immune complex and is not merely an artifact of the PEG precipitation, since this band is also obtained when the immune complex is further treated with the antibody-binding beads (Lane 2). The results obtained using PEG precipitation of the immune complex are more variable and less quantitative than those obtained when the PEG precipitation step is combined with the use of antibody-binding beads. In many instances, the use of PEG precipitation alone does not result in a detectable antigen band (as detected by Western blot), while use of the present method does enable detection of the antigen band.

In addition to the detection of OspA, the methods disclosed herein may also be used to detect the presence of other B. burgdorferi cell surface antigens, such as OspB, OspC, OspD, OspE, OspF and OspG, as well as other cell surface antigens known to those in the art.

### EXAMPLE 2

Enzyme-linked IgM Capture Immune Complex Biotinylated Antigen Assay (EMIBA)

Samples were obtained from three patient populations. In the first set, serum samples were obtained from 131 patients evaluated at the Lyme Disease Center at RWJMS for possible Lyme disease. Before testing in our laboratory and based solely on clinical examination, the patients were designated as having a) “active” Lyme disease, b) prior Lyme disease without evidence of current active infection, and c) no evidence of prior or present Lyme disease. The results of the clinical evaluation were unknown to the laboratory personnel performing the assays. Patients were considered to have “active infection” if they had present or previous erythema migrans or objective features of early disseminated (carditis or neurologic features, including lymphocytic meningitis, cranial nerve palsy or radiculoneuritis) or late (arthritis or tertiary neuroborreliosis) Lyme disease and had not yet received an adequate course of antibiotic therapy for their Lyme disease. Patients who had received appropriate antibiotic therapy and, at the time of phlebotomy, had no evidence of inflammatory, skin, heart, joints or peripheral nervous system or central nervous system were identified as having “prior” infection. All samples were tested by RWJMS University Diagnostics Laboratory's Lyme Disease Laboratory using IgG or IgM isotype-specific ELISA and immunoblot kits (MarDx, Carlsbad, Calif.) following the manufacturer's directions (1:100 dilution). In some studies, IgM immunoblots using serum at 1:10 dilution were compared with immune complexes (1:10 dilution optimal) following the manufacturer's instructions. Use of blood samples was approved by the RWJMS IRB (Protocol W-0093). At the time of evaluation and phlebotomy, 64 of the 131 patients from the Lyme Disease Center were clinically designated as having “active” disease (diagnosed with Lyme disease but received no prior antibiotic treatment); 28 had “prior” Lyme disease (all previously antibiotic treated, with no evidence of active Lyme disease at the time of phlebotomy); and 39 never had Lyme disease.

The second set of Lyme disease serum samples (totaling 42) were obtained from a blinded Centers for Disease Control and Prevention collection (Bacterial Zoonoses Branch, CDC, Atlanta, Ga.). These samples had been previously tested by commercial kit IgG or IgM ELISA and immunoblots (MarDx Diagnostics, Carlsbad, Calif. (hereinafter “MarDx”)); and CDC's flagellin-enriched IgM and IgG ELISA. Clinical information and the results of testing were supplied after completion of these experiments. Based on the clinical information provided by the CDC after laboratory testing was completed, 38 patients could be assigned to “active” or “prior” groups, using the above criteria.

The third set of Lyme disease serum samples (totaling 11) were from patients with EM biopsy culture-proven B. burgdorferi infection. Sera had previously been tested using IgM IFA; IgM ELISA; IgM immunoblot; and polyvalent ELISA. The results of these analyses were withheld until completion of EMIBA experiments. Sera were obtained at time of biopsy, before treatment with antibiotics. All patients exhibited active Lyme disease.

In addition to the three sets of Lyme disease serum samples, sera from patients having various autoimmune conditions were also tested. Among these were sera from multiple sclerosis patients (a total of 12 patients); from VDRL positive sera (titers between 1:2 and 1:256) from patients with syphilis (a total of 22 samples); sera from patients with inflammatory joint disease (5 with systemic lupus erythematosus, 8 with rheumatoid arthritis and 2 with gout). Two blinded VDRL-negative controls were included with VDRL-positive sera collection.

Table 1 is the results of EMIBA, “free antibody” assay, IgM and IgG isotype-specific ELISA and IgM and IgG isotype-specific immunoblot assays in 131 patients evaluated at the Lyme Disease Center for possible Lyme disease. Of the 131 patients, 64 had active Lyme disease at the time of phlebotomy; 28 had evidence of “prior” Lyme disease but not active infection at the time of phlebotomy and 39 had no evidence of present or past Lyme disease.

TABLE 1

| | | 95% CI^{a} | | 95% CI for |
|---|---|---|---|---|
| | Sensitivity | for Sensitivity | Specificity | Specificity |
| Assay Type | (%) | (%) | (%) | (%) |
| EMIBA^{b} | 98 | 90-100 | 96 | 87-99 |
| Free antibody^{c} | 95 | 86-99 | 91 | 81-96 |
| IgM ELISA | 66 | 53-77 | 76 | 64-86 |
| IgG ELISA | 58 | 45-70 | 87 | 76-93 |
| IgM | 58 | 45-70 | 84 | 72-91 |
| immunoblotting | | | | |
| IgG | 44 | 32-57 | 93 | 83-97 |
| immunoblotting | | | | |

^{a}CI = confidence interval
^{b}Assay detects antibodies derived from serum ICs
^{c}Assay is identical to EMIBA except for use of serum in place of ICs

EMIBA results correlated better with clinical findings than the “free antibody” assay, although the difference was not statistically significant (Table 1). Both EMIBA and the “free antibody” assay were significantly more sensitive than all other assays (p<0.001); for each comparison with the other assays, EMIBA was slightly superior to the “free antibody” assay (Table 2).

TABLE 2

| | Difference in | | 95% CI^{a} for | | Difference in | | 95% CI for | |
|---|---|---|---|---|---|---|---|---|
| | sensitivity | | sensitivity (%) | | specificity (%) | | specificity (%) | |
| Assay Type | EMIBA^{b} | FA^{c} | EMIBA | FA | EMIBA | FA | EMIBA | FA |
| FA | NS | —^{d} | | | NS^{e} | − | | |
| IgM ELISA | 33 | 30 | 20-46 | 17-43 | 19 | 15 | 6-32 | 4-26 |
| IgG ELISA | 40 | 38 | 27-53 | 25-51 | NS | NS | | |
| IgM | 40 | 38 | 27-53 | 25-51 | 12 | NS | 3-21 | |
| immunoblotting | | | | | | | | |
| IgG | 55 | 52 | 40-70 | 39-65 | NS | NS | | |
| immunoblotting | | | | | | | | |

^{a}CI = confidence interval
^{b}Assay detects antibodies derived from serum ICs
^{c}FA = free antibody assay. Assay is identical to EMIBA except for use of serum in place of ICs
^{d}No comparison of FA with itself
^{e}NS = not statistically significant

EMIBA was slightly more specific than the “free antibody” assay (Table 1), and both were more specific than the other assays (Table 2). Some of these differences were statistically significant: EMIBA was more specific than IgM ELISA (p<0.002) and IgM immunoblot (p=0.022), while the “free antibody” assay was superior only to the IgM ELISA (p=0.012). IgG immunoblot was as specific as either EMIBA or the free antibody assay.

After unblinding the CDC serum collection we calculated sensitivity and specificity in the 38 samples that could be designated as being from “active” or “prior” patients (Table 3). EMIBA and the “free antibody” assay were 100% sensitive, somewhat better than all but the CDC assay; these differences were not statistically significant.

TABLE 3

| | | 95% CI^{a} | | 95% CI for |
|---|---|---|---|---|
| | Sensitivity | for Sensitivity | Specificity | Specificity |
| Assay Type | (%) | (%) | (%) | (%) |
| EMIBA^{b} | 100 | 63-99 | 66 | 46-81 |
| Free antibody^{c} | 100 | 63-99 | 48 | 30-66 |
| IgM ELISA | 78 | 40-96 | 43 | 25-63 |
| IgG ELISA | 78 | 40-96 | 57 | 39-75 |
| IgM | 56 | 23-85 | 62 | 42-79 |
| immunoblotting | | | | |
| IgG | 89 | 51-99 | 59 | 39-76 |
| immunoblotting | | | | |
| CDC ELISA^{d} | 100 | 63-99 | 17 | 7-36 |

^{a}CI = confidence interval
^{b}Assay detects antibodies derived from serum ICs
^{c}Assay is identical to EMIBA except for use of serum in place of ICs
^{d}Flagellin-enhanced ELISA developed by the Centers for Disease Control

The EMIBA had a higher specificity than the “free antibody” assay, a difference significant at p<0.05. The sensitivities of both experimental assays were significantly superior to the flagellin-enhanced CDC ELISA for both comparisons (p<0.001) (Table 3). Thus, in a blinded, independent well-defined serum collection, EMIBA and the “free antibody” assay, out-preformed the other assays. For all assays, specificities were lower in analysis of the CDC serum collection than in studies of the Lyme Disease Center samples (Table 1).

EMIBA detection of serologic reactivity in EM (early Lyme disease) was compared with standard serodetection assays. Twenty-seven of 131 samples from the Lyme Disease Center were from patients with EM. Of these, 24 samples were obtained before treatment. The remaining three were from patients who had been successfully treated with antibiotics 3 to 9 months before phlebotomy; none of the patients had evidence of infection at the time of phlebotomy. These three samples constitute the TN and FP groups in Table 4.

TABLE 4

| | Sensitivity | Specificity |
|---|---|---|
| Assay Type | (%) | (%) |
| EMIBA | 100 | 100 |
| Free antibody | 96 | 100 |
| IgM ELISA | 58 | 67 |
| IgG ELISA | 54 | 100 |
| IgM immunoblotting | 58 | 33 |
| IgG immunoblotting | 42 | 67 |

Referring now to Table 4, EMIBA and the “free antibody” assay were more sensitive than comparison assays and more specific than all but IgG ELISA, to which they were equivalent. Thus, EMIBA and the “free antibody” assay were superior to other assays in corroborating early Lyme disease.

Within the CDC panel were 28 samples from EM patients (Table 5). EMIBA, the “free antibody” assay, IgM ELISA and the flagellin-enhanced CDC ELISA were 100% sensitive, with IgG ELISA being the least sensitive assay. The specificities of EMIBA, IgG ELISA and IgG immunoblot were comparable, but the specificity of the CDC ELISA was substantially lower than the other tests. In sera from the 11 patients with culture-positive EM from the Marshfield Clinic, eight samples were positive as tested by EMIBA; seven samples were positive as tested by the “free antibody” assay; seven samples were positive as tested by the IgM IFA; three samples were positive as tested by the IgM EIA; four samples were positive as tested by the IgM immunoblot; and three samples were positive as tested by the polyvalent EIA. EMIBA was positive in 3 IFA-negative samples; IFA was positive in 2 EMIBA-negative specimens. The “free antibody” assay did not detect seroreactivity in any EMIBA or IFA negative sera. In only 1 of the samples were both EMIBA and the “free antibody” assay negative. Thus, in culture-verified EM patients, EMIBA was superior to the other assays.

TABLE 5

| | Sensitivity | Specificity |
|---|---|---|
| Assay Type | (%) | (%) |
| EMIBA | 100 | 73 |
| Free antibody | 100 | 55 |
| IgM ELISA | 100 | 33 |
| IgG ELISA | 67 | 71 |
| IgM immunoblotting | 83 | 50 |
| IgG immunoblotting | 83 | 77 |
| CDC ELISA | 100 | 23 |

The EMIBA was investigated to determine its ability to differentiate between seropositivity in patients with ongoing infection and persisting seropositivity related to past infection (“active” vs. “prior” disease). Of the 131 Lyme Disease Center patients, 64 had active infection and 28 had previous Lyme disease without evidence of active disease at the time of phlebotomy, i.e. previously cured Lyme disease (Table 6). Table 6 presents serologic results in the comparator assays with reference to results in EMIBA and the “free antibody” assay.

TABLE 6
Results for patients with active disease

| No. of patients | | | ELISA | | Immunoblot | |
|---|---|---|---|---|---|---|
| (total 64) | FA | EMIBA | IgM | IgG | IgM | IgG |
| 2 | +^{a} | −^{b} | 1 | 1 | 0 | 0 |
| 1 | − | + | 0 | 0 | 1 | 1 |
| 61 | + | + | 41 | 36 | 36 | 27 |
| 0 | − | − | 0 | 0 | 0 | 0 |

Results for patients with prior disease

| No. of patients | | | ELISA | | Immunoblot | |
|---|---|---|---|---|---|---|
| (total 28) | FA | EMIBA | IgM | IgG | IgM | IgG |
| 1 | + | − | 1 | 1 | 1 | 1 |
| 3 | − | + | 3 | 1 | 2 | 1 |
| 1 | + | + | 0 | 0 | 0 | 0 |
| 23 | − | − | 8 | 3 | 8 | 3 |

^{a}+ = antibodies detected (positive result)
^{b}− = no antibodies detected (negative result)

EMIBA was positive in 62 of 64 samples of patients with active Lyme disease and the “free antibody” assay was positive in 63 of 64 samples. The other assays were less often positive in patients with active and ongoing infection. Only the IgM ELISA was positive in nearly two-thirds of active patients and IgG immunoblot was positive in only 44%. In the 2 EMIBA-negative samples neither isotype-specific immunoblot was positive.

In 24 of 28 “prior” patients EMIBA was negative; the “free antibody” assay was negative in 26 of 28. In these patients without active infection, between 5 and 12 samples were positive in each of the other assays. Thus, EMIBA and the “free antibody” assay performed better in samples from patients with “active” and “prior” infection than the other assays.

Of the 38 assignable CDC samples, nine were designated as being from patients with active infection and 29 were from patients with prior Lyme disease. The results in Table 7 are presented in the same manner as in Table 6. All nine of the CDC samples from patients with active infection were EMIBA and “free antibody” assay positive (Table 7); of the other assays only the CDC ELISA was positive in all patients. In patients with “prior” disease the assay that is most often negative would be the best at differentiating “prior” from “active” disease (a positive result in a person with infection that is no longer active is, in essence, a “false positive”). Fourteen of the 29 samples from patients with “prior” disease were negative by EMIBA. Nineteen of the “free antibody” assay results were negative. IgM immunoblot was negative in 18 samples, IgG ELISA and immunoblot in 17 each, IgM ELISA in 13 and the CDC ELISA in only five.

TABLE 7
Results for patients with active disease

| No. of patients | | | | ELISA | | Immunoblot | |
|---|---|---|---|---|---|---|---|
| (total 9) | FA | EMIBA | CDC ELISA | IgM | IgG | IgM | IgG |
| 0 | +^{a} | −^{b} | 0 | 0 | 0 | 0 | 0 |
| 0 | − | + | 0 | 0 | 0 | 0 | 0 |
| 9 | + | + | 9 | 7 | 7 | 5 | 8 |
| 0 | − | − | 0 | 0 | 0 | 0 | 0 |

Results for patients with prior disease

| No. of patients | | | | ELISA | | Immunoblot | |
|---|---|---|---|---|---|---|---|
| (total 29) | FA | EMIBA | CDC ELISA | IgM | IgG | IgM | IgG |
| 0 | + | − | 0 | 0 | 0 | 0 | 0 |
| 5 | − | + | 5 | 2 | 3 | 1 | 3 |
| 10 | + | + | 9 | 8 | 5 | 6 | 6 |
| 14 | − | − | 10 | 6 | 4 | 4 | 3 |

^{a}+ = antibodies detected (positive result)
^{b}− = no antibodies detected (negative result)

Thus, the “free antibody” assay agreed with the clinical status better than other assays (9/9 TP and 19/29 TN); while the CDC ELISA was least likely to predict clinical status (9/9 TP and only 5/29 TN). The other assays were not remarkably different and all were in general agreement with clinical status: IgG immunoblot (8/9 TP and 17/29 TN); EMIBA (9/9 TP and 14/29TN) and IgM immunoblot (5/9 TP and 18/29 TN); IgG ELISA (7/9 TP and 16/29 TN); and IgM ELISA (7/9 TP and 12/29 TN).

Eleven of 29 “prior” patients had been successfully treated with antibiotics three months or less prior to phlebotomy; eight of these 11 samples were EMIBA “false positive” results. Persistence of immune complex in these samples may represent ongoing immune complex formation during recently active infection, so that all immune complex had not yet been cleared from the circulation. As noted above, eight of 11 culture-verified EM samples were positive as tested by EMIBA; in six of these eight, the “free antibody” assay was also positive.

The EMIBA is also useful to accurately diagnose Lyme disease in patients suffering from other diseases, such as multiple sclerosis, syphilis and rheumatic diseases, that could be confused with Lyme disease. For example, flagellin antigen, present in syphilis patients, produces false positive results in many Lyme tests. The symptoms of multiple sclerosis and rheumatic disease are easily confused with those of Lyme disease. One of the 12 multiple sclerosis patients'sera tested was positive for both free antibodies and immune complex by EMIBA; the sample was negative by IgM immunoblot. In the “free antibody” assay 55% (12/22) of VDRL-positive samples were reactive, whereas 32% (7/22) were positive as tested by EMIBA. All EMIBA-positive samples were also “free antibody” positive, i.e. the EMIBA-positives were a sub-set of the “free antibody” positive group. Of the 12 “free antibody” positive sera, six were positive by standard B. burgdorferi IgM immunoblot. Six of the seven EMIBA-positive samples were positive by standard IgM immunoblot.

All five samples positive in the “free antibody” but negative in EMIBA were negative by immunoblot. Both VDRL-negative controls were negative in both EMIBA and the “free antibody” assay. Thus, EMIBA was superior to the “free antibody” assay (was positive in fewer syphilis sera) and in six of seven EMIBA-positive samples detected IgM cross-reacting with B. burgdorferi proteins with sufficient affinity to produce a positive anti-B. burgdorferi immunoblot. Of the 15 active inflammatory rheumatologic disease patients tested none was positive in either the “free antibody” assay or EMIBA.

Only with highly sensitive and specific serological or microbiological tests can unusual or atypical clinical features of B. burgdorferi infection be correctly included within the spectrum of Lyme disease and illusory associations be identified as such and excluded. Many serological assays are currently available to detect anti-B. burgdorferi antibodies, but all share two limitations: 1) in early disease the tests may not detect low levels of specific antibody; and 2) none differentiates persistent seropositivity in active infection from clinically irrelevant persisting antibodies, i.e. seropositivity without evidence of active infection. The current studies represent the largest and broadest application of immune complex technology to the seroconfirmation of Lyme disease and the only comparison of IC-based assays with “free antibody”-based assays and currently available immunoassays (IFA, ELISA, flagellin-enhanced ELISA and Western blot). It is known that anti-B. burgdorferi IgM may persist in the serum of patients with Lyme disease after treatment and apparent cure. In order to detect even very low IgM levels in ver early or later disease we made use of the increased sensitivity intrinsic to EMIBA; we were able to further enhance sensitivity by biotinylating the sonicate, giving the same amplification benefits as in biotin-enhanced immunoblot.

Lyme disease sera in this study were from three different sources and in all cases the testing was done blinded to clinical information. The first group of sera was from patients evaluated for Lyme disease at a Lyme disease regional referral center. This population is the most relevant to a serologic trial—patients from an endemic area whose symptoms and signs prompted the patients and/or their physicians to consider Lyme disease. The second group was from the CDC collection, an established serum bank used in previous serologic studies. The third group, from the Marshfield Clinic, represents an incontrovertible “gold standard” for early Lyme disease—culture-positive EM patients, with normal controls interspersed.

Studies using all three serum banks showed EMIBA can serologically confirm early Lyme disease. EMIBA and, to a lesser degree, the “free antibody” assay were superior to the comparator assays. In the Lyme Disease Center sera, EMIBA was better able to differentiate between persisting seropositivity indicating active infection (thereby warranting antibiotic therapy) and seropositivity of no clinical significance. EMIBA was somewhat less effective in the CDC sera. This was most apparent in all the assays'ability to differentiate “active” from “prior” disease.

Disease status was assigned in 38 CDC samples; clinical information was less detailed than that available for the Lyme Disease Center samples, especially concerning the timing of previous treatment and time between treatment and phlebotomy. Differences in the correlation of the EMIBA data with disease status may relate to improper assignment of patient samples to “active” or “prior” groups, possibly based on incomplete information and/or the fact that a short time had elapsed from successful treatment to phlebotomy. In the latter case, false positive EMIBA results might be due to persistence of immune complex following treatment. It is also possible that the smaller size of the CDC bank may have obscured differences in the predictive power of the EMIBA.

Sera from patients with syphilis contain antibodies that also bind to B. burgdorferi. In this control group EMIBA was superior to the “free antibody” assay, in that fewer of the luetic sera were positive. False positive results were present in other assays, as well—six of the seven EMIBA-positive samples were positive in IgM immunoblot. A small proportion of multiple sclerosis patients also had antibodies reacting with B. burgdorferi proteins. Efforts are under way to reduce the occurrence of false-positive results, especially in these groups.

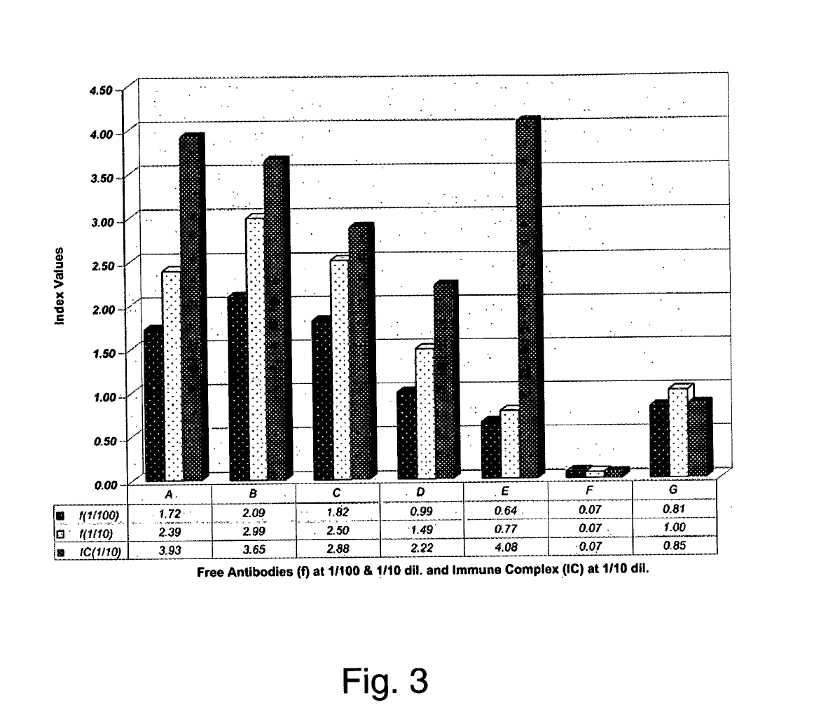

FIG. 3 shows results from IgM capture ELISA that detected anti-Borrelia burgdorferi IgM, and compares the index values of a 1:100 free antibody dilution and a 1:10 dilution of immune complexes. For samples A to E (representative of patients tested), the index value is higher for the immune complexes. Results for a 1:10 immune complex dilution are higher than those for a 1:10 free antibody dilution. Total IgM was measured in both immune complex and free antibody samples from Lyme disease and non-Lyme disease samples by quantitative IgM capture ELISA and confirmed by commercial laboratory nephelometry. There was a 3- to 7-fold greater total IgM in free antibody than in immune complex in all sample pairs; the greater reactivity in immune complexes in the direct comparisons of 1:10 dilutions in immune complexes and free antibody occurred in spite of there being a lower IgM concentration in the immune complex fraction. The negative control (sample F) had negative results for both immune complexes and free antibody at both concentrations. Surprisingly, the immune complexes of sample G (from a patient who had previously been treated and cured of Lyme disease) did not contain increased levels of specific antibodies.

EMIBA is slightly superior to the “free antibody” assay; superior to the standard assays in sero-confirmation of Lyme disease; effective in detecting antibodies in early disease; better than standard assays and comparable to IFA in the small group of sera from patients with positive skin-biopsy cultures; and helpful in determining the clinical significance of seropositivity. EMIBA compared favorably with commercial and CDCP flagellin-enhanced ELISA and other assays; EMIBA more accurately confirmed early disease and active ongoing infection. Thus, EMIBA is a promising new assay for accurate serological confirmation of early and/or active Lyme disease. IC-based serological assays are valuable in sero-confirmation of the diagnosis and clinical status of B. burgdorferi infection and might be useful in other diseases, as well. Such diseases are those characterized by the presence of immune complexes, such as, for example, autoimmune diseases (rheumatoid arthritis, Sjogren's syndrome, progressive systemic sclerosis, mixed connective tissue disease, polyarteritis nodosa, glomerulonephritis), and various infectious diseases (i.e., acquired immune deficiency syndrome (AIDS), infective endocarditis, disseminated streptococcal, staphylococcal, meningococcal, or gonococcal infections, syphilis, leprosy, hepatitis B, cytomegalovirus infection, infectious mononucleosis, malaria, toxoplasmosis, or trypanosomiasis).

In early disease, a state of antigen excess, the IgM of the humoral response to a new pathogen may be sequestered in IC, bound to pathogen- or transformed cell-derived antigen targets and undetectable by standard serum-based assays. In such circumstances “free antibody” levels would be undetectable. The ongoing production of Immune complexes is dependent on, and limited by, the ongoing production of pathogen- (or transformed cell-) derived antigens, so IC-based assays might be useful in determining if persisting seropositivity is a marker of disease persistence following treatment for infectious or malignant diseases. EMIBA can replace the current two-tiered serological confirmation approach in Lyme disease, with a single assay.

Various patents and publications are cited herein, and their disclosures are hereby incorporated by reference in their entireties. The present invention is not intended to be limited in scope by the specific embodiments described herein. Although the present invention has been described in detail for the purpose of illustration, various modifications of the invention as disclosed, in addition to those described herein, will become apparent to those of skill in the art from the foregoing description. Such modifications are intended to be encompassed within the scope of the present claims.

What is claimed is:

1. A method for detecting an antigen present in an immune complex, comprising:
a. isolating an immune complex from a bodily sample derived from a patient, said immune complex including an antigen and an antibody directed against said antigen;
b. incubating said immune complex under conditions effective to dissociate said immune complex and separate said antigen from said antibody;
c. incubating said dissociated immune complexes under conditions effective to reassociate said antigen and said antibody;
d. incubating said reassociated antigen and antibody with at least one member selected from the group consisting of protein A-agarose beads, protein G-agarose beads, protein L-agarose beads, mannan-binding protein-agarose beads, and agarose beads containing at least two of protein A, protein G, protein L and mannan-binding protein, under conditions effective to permit binding of said antibody by said at least one member;
e. separating said at least one member from said solution; and
f detecting the presence of said antigen through a binding reaction specific for said antigen.

2. The method of claim 1, wherein said antigen is specific for Lyme disease.

3. The method of claim 1, wherein said antigen is a cell surface antigen of Borrelia burgdorferi.

4. The method of claim 3, wherein said antigen is a member selected from the group consisting of OspA, OspB, OspC, OspD, OspE, OspF and OspG.

5. The method of claim 3, wherein said antigen is OspA.

6. The method of claim 1, wherein said antigen is specific for an infectious disease.

7. The method of claim 6, wherein said infectious disease is a member of the group consisting of acquired immune deficiency syndrome, Lyme disease, infective endocarditis, streptococcal infection, staphylococcal infection, meningococcal infection, gonococcal infection, syphilis, leprosy, hepatitis B, cytomegalovirus infection, infectious mononucleosis, malaria, toxoplasmosis, and trypanosomiasis.

8. The method of claim 1, wherein said antigen is specific for an autoimmune disease.

9. The method of claim 8, wherein said autoimmune disease is a member of the group consisting of rheumatoid arthritis, Sjogren's syndrome, progressive systemic sclerosis, mixed connective tissue disease, polyarteritis nodosa, and glomerulonephrritis.

10. The method of claim 1, wherein said immune complex is isolated by incubation with a solution containing polyethylene glycol.

11. The method of claim 1, wherein said member in step d) consists of protein G and mannan-binding protein.

12. The method of claim 1, wherein said member in step d) consists of mannan-binding protein.

13. The method of claim 1, wherein said binding reaction of step e) is performed on a solid phase bearing a binding agent specific for said antigen.

14. The method of claim 14, wherein said solid phase is a dipstick or a slide.

15. A method for detecting an antibody present in an immune complex, comprising:
a. isolating an immune complex from a bodily sample derived from a patient, said immune complex including an antigen and an antibody directed against said antigen;
b. incubating said immune complex under conditions effective to dissociate said immune complex and separate said antigen from said antibody;
c. incubating said dissociated immune complexes with a solid phase bearing an anti-IgM antibody under conditions effective to permit binding of said antibody of step b) by said anti-IgM antibody;
d. incubating said solid phase with a biotinylated sonicate of Borrelia burgdorferi cells; and
e. detecting the presence of said antibody bound to said solid phase through a binding reaction specific for said biotinylated sonicate.

16. The method of claim 15, wherein said binding reaction of step e) uses a molecule containing avidin, streptavidin, or a derivative thereof.

17. The method of claim 15, wherein said binding reaction of step e) uses a molecule containing an anti-biotin antibody or a derivative thereof.

18. The method of claim 15, wherein said solid phase is in the form of a plate.

19. The method of claim 15, wherein said solid phase is in the form of agarose beads.
